(19) United States
(12) Patent Application Publication
(10) Pub. No.: US 2023/0113383 A1
(43) Pub. Date: Apr. 13, 2023

(54) Augmented Reality Guidance System For Guiding Surgical Operations On An Articulating Portion Of A Bone

(71) Applicants: One Ortho, SAINT-GENIS-LAVAL (FR); Franco-Swiss Orthoconsulting, OBERHAUSBERGEN (FR)

(72) Inventors: Jean-François GONZALEZ, TOULON (FR); Bertrand COULET, SAINT-GELY-DU-FESC (FR); Christophe ALEPEE, LYON (FR); Nicolas HOLZER, GRAND LANCY (CH)

(73) Assignees: One Ortho, SAINT-GENIS-LAVAL (FR); Franco-Swiss Orthoconsulting, OBERHAUSBERGEN (FR)

(21) Appl. No.: 17/796,130

(22) PCT Filed: Dec. 21, 2020
(86) PCT No.: PCT/FR2020/052581

§ 371 Date: Jul. 28, 2022

(30) Foreign Application Priority Data
Feb. 20, 2020 (FR) 2001685

(51) Int. Cl. A61B 34/20; A61B 17/17

(52) U.S. Cl. CPC A61B 34/20; A61B 17/1703; A61B 2034/2057; A61B 2034/2065; A61B 2090/3983

(57) ABSTRACT

An augmented reality guidance system for guiding surgical operations on an articulating portion of a bone includes a tracking element to be attached to the articulating portion and-having a surface bearing against the portion that is custom-designed from a virtual model , and a three-dimensional reference mark ; a probe with a three-dimensional reference mark for probing at least three regions of a portion apart from the cartilage area; a camera for displaying and tracking the reference marks ; and a processing system for processing virtual bone model data and real-time image data from the camera to determine the position and relative direction of the tracking element and probe, to display on a display device , a three-dimensional virtual representation of a geometric datum relating to the bone model having position and direction determined by the position and direction of the tracking device and corrected after probing.

### TECHNICAL FIELD

The present invention relates to the technical field of orthopaedic surgery, and more particularly to an augmented reality system for guiding surgical operations on an articulating portion of a bone.

The invention is of application to assisting the positioning of any type of orthopaedic joint implant, such as knee, shoulder or hip prostheses and, more generally, any type of orthopaedic implant requiring cuts or orifices to be made in the zone of the anatomical bone zone that is to be fitted with a prosthesis.

### PRIOR ART

It is well known for a person skilled in the art to use different devices, generally known as insertion ancillaries, for the placement of different orthopaedic implants. As an example, the devices may by constituted by tibial guide blocks, femoral guide blocks, etc. These blocks incorporate slots and orifices in their thickness for the passage and guidance of instruments with a cutting blade or drill bit, the slots or orifices being suitably oriented and positioned as a function of the bone cuts or drilling which has to be carried out for implantation of the implant.

It is also known that these guide blocks comprise at least one face for bearing on the articulating portion, made to measure on the basis of a previously established virtual model.

In this manner, during the intervention, the surgeon positions the tailor-made guide on the bone, which guide must theoretically cooperate in a single position and orientation with the portion of the bone in order to be able to guide the cutting or drilling operations carried out by the surgeon in an optimal manner.

However, in practice, precise positioning of this made to measure guide is still difficult.

Document FR 3 078 624 has already proposed attempting to overcome this disadvantage by proposing an augmented reality assistance system for positioning specific surgical instrumentation in a patient.

According to that document, the system comprises:
a guide block intended to be attached to the articulating portion of the bone and comprising, on the one hand, at least one face for bearing on the bone, made to measure from a virtual bone model, and on the other hand a three-dimensional visual marker;
a camera for visualizing and tracking the marker;
a processing system configured to process data from the virtual bone model and, in real time, image data obtained from the camera in order to determine the position and orientation of the guide block, in such a manner as to display, on the display unit secured to the camera, and superimposed on the field of view of a user, a three-dimensional virtual representation of at least one geometric datum relating to the bone model the position and orientation of which are determined by those of the guide block.

In other words, according to that document, it is possible to visualize a mechanical axis or contours of the bone superimposed on real images of the guide block.

This assists the surgeon in correctly positioning the guide block during the operation. In practice, the surgeon uses the virtual representations of the mechanical axis of the bone and the contours, for example of the epiphysis, to correctly position the guide block, by matching and superimposing said virtual representations on the real bone.

This technique is interesting, but relies on the surgeon’s ability to make the virtual information coincide with the real information, which could lead to a risk of error.

In fact, during the surgical operation on a bone articulation, only said articulation is visible to the surgeon. The other parts of the limb comprising the articulation are masked by the surgical field, and the articulating itself is stained with blood, and might be covered with fat, which complicates the operation.

Furthermore, and importantly, when the virtual bone model is obtained by techniques of the scanner type, for example, these techniques do not take the cartilage of the bone into account. MRI makes it possible to take this cartilage into account, but this technique is stressful for the patient, expensive, and has relatively long waiting times.

In this manner, the virtual bone model on the basis of which the guide is made to measure is inaccurate. The guide block has been custom designed to fit over a bone which does not include cartilage.

As a result, when the surgeon is going to attempt to position the guide block on the portion of the articulation to be operated on, the cartilage will create an extra thickness which will accordingly offset the cutting planes and drilling axes planned and positioned by the guide block.

Thus, in addition to the difficulty in finding the correct positioning of the made to measure guide, which therefore no longer fits the bone very accurately, this also results in inaccuracies in respect of positioning the various cutting planes and drilling axes.

To overcome this problem, the surgeon may decide to retract the cartilage, but this solution is not optimal since retraction of the cartilage cannot be controlled very well.

Another solution is to produce a made to measure guide which comes to bear on zones of the bone where there is no cartilage. However, this technique suffers from the disadvantage of providing very bulky guides, and therefore the surgery is more invasive and runs a greater risk of infection.

Document WO 2019/141704, which describes an augmented reality system for guiding a surgical operation on an articulating portion of a bone, is also known. However, this document remains silent in respect of the difficulties represented by the presence of cartilage to correctly position the tracking elements. WO 2019/141704 describes the presence of two tracking elements, attached to the bone. WO 2019/141704 therefore involves making an additional incision and providing additional anchorage for securing the second tracking element, which presents disadvantages for the patient.

### DISCLOSURE OF THE INVENTION

One of the aims of the invention is to provide a system for guiding the various operations of cutting or piercing a bone joint, the accuracy of which is optimal, while remaining simple, quick to carry out, with a limited risk of infection.

To this end, an augmented reality system has been developed for guiding a surgical operation on an articulating portion of a bone, the system comprising:
a tracking element intended to be attached to the articulating portion of the bone, the tracking element comprising, on the one hand, at least one face for bearing on the articulating portion made to measure from a virtual model of the articulating portion of the bone and, on the other hand, a three-dimensional visual marker;
a camera for visualizing and tracking the marker;
a processing system configured to process data from the virtual bone model and, in real time, image data obtained from the camera in order to determine the position and orientation of the tracking element in such a manner as to display, on a display unit secured to the camera and superimposed on the field of view of a user, a three-dimensional virtual representation of at least one geometric datum relating to the bone model the position and orientation of which are determined by the position and orientation of the tracking element.

In accordance with the invention, the guidance system comprises a sensor which is also provided with a three-dimensional visual marker and intended to palpate at least three zones of a portion of the bone, outside the cartilage zone. The marker of the sensor is also intended to be tracked by the camera, and the processing system is further configured in order to determine the position and orientation of the sensor with respect to the position and orientation of the tracking element in a manner such as to correct, on the display unit and after palpation, the position and orientation of the three-dimensional virtual representation of the geometric datum relating to the bone model with respect to the tracking element.

In this manner, the invention makes it possible to position a tracking element on the articulating portion of the bone, the position of which is known and marked with optimum precision.

In fact, the essence of the invention is not to position a guide block directly, but simply to position a tracking element which carries a marker the position of which is precisely known with respect to the virtual bone model.

Thus, during the intervention, the surgeon places the tracking element on the bone, with inaccuracy related to the cartilage. The surgeon then picks up the sensor provided with a marker, and starts to palpate zones, for example at least three zones of the bone, outside the cartilage area. By correlating the palpated zones with those of the virtual bone model, this operation will enable the position and the orientation of the virtual representation of the geometric datum relating to the virtual model with respect to the real position of the bone to be readjusted precisely.

The geometric datum relating to the bone model represented virtually and displayed on the display unit may be of any type, such as a geometric surface or contours of the bone model, a mechanical axis, planned cutting planes or drilling axes, etc.

The palpation technique, also known by the English expression “bone morphing”, is carried out here on only three zones, and therefore is not of a nature which is too invasive, nor does it carry the risk of infection which is known in this technique.

In fact, unlike the “bone morphing” technique of the prior art, it is not necessary to attach pins to the bone in question with incisions that are not justified by the surgical procedure. The risk of infection is thus not increased, and the intervention time is not extended.

The present invention therefore makes it possible to attach a visual marker to the articulating portion, the precise position of which is known, which position has been readjusted specifically with respect to the real position of the bone.

This makes it possible to assist the use of other objects, such as guide blocks or instruments, which are themselves marked, their relative position being accurately known with respect to the tracking element and thus with respect to the displayed virtual representation of the geometric datum relative to the bone model which, itself, will be perfectly positioned with respect to the real bone.

To this end, the system comprises, for example, a cutting or drilling instrument provided with a three-dimensional visual marker; the marker of the instrument is also intended to be tracked by the camera, and in addition, the processing system is configured in order to determine the position and the orientation of the instrument with respect to the position and the orientation of the tracking element in a manner such as to display, on the display unit, the position and the orientation of a cutting plane or drilling axis linked to the instrument with respect to the tracking element.

Several embodiments of the display unit may be envisaged without departing from the scope of the invention.

As an example, the display unit may be a screen displaying the images obtained from the camera, or in fact it may be in the form of at least one spectacle lens, the spectacles optionally carrying the camera, and intended to be worn by the user, the display unit displaying only the virtual representations.

Advantageously, the geometric datum relating to the bone model corresponds to the external surface of said virtual bone model, the processing system being further configured, after correction, to process the data for the virtual bone model in order to increase its dimensions at the level of the bearing surface of the tracking element so that the display unit represents the bearing face of the tracking element in contact with the external surface of the virtual bone model.

In accordance with one embodiment, the tracking element comprises means for securing a guide block, or in fact comprises at least one orifice for guiding a pin for securing to the bone, and a guide block having at least one orifice for passage of the pin, the guide block being intended to be secured to the bone in a position and at an orientation which is determined and guided by the pin. The term “guide block” should be understood to mean a block which comprises at least one slot or at least one orifice in order to form at least one cutting or drilling guide.

Advantageously, the guide block comprises at least one slot and/or at least one guide orifice, the position and/or the orientation of which with respect to the body of the guide block can be adjusted.

Preferably, and in order to facilitate the surgical operation, the visual marker is attached to the tracking element in a removable or separable manner. Thus, when the tracking element has made it possible to correctly position a guide block, or to directly position cutting or drilling instruments, the visual marker of the tracking element can be broken off or removed so as not to impede the cutting or drilling operations per se.

### DESCRIPTION OF THE FIGURES

Other features and advantages of the invention will become apparent from the following description, which is given by way of non-limiting indication and made with reference to the accompanying figures, in which:

FIG. 1 is a diagrammatic perspective view illustrating a tracking element of the system in accordance with the invention, positioned on an articulating portion of a femur.

FIG. 2FIG. 2 is a diagrammatic view illustrating the display unit, processing system, and the camera of the augmented reality guidance system of the invention.

FIG. 3FIG. 3 is a diagrammatic perspective view illustrating a tracking element of the system in accordance with the invention, positioned on an articulating portion of a femur, on the hip side.

FIG. 4FIG. 4 is a diagrammatic perspective view illustrating a tracking element of the system in accordance with the invention positioned on an articulating portion of a humerus, on the shoulder side.

FIG. 5FIG. 5 is a diagrammatic perspective view illustrating a tracking element of the system in accordance with the invention, positioned on an articulating portion of a scapula.

FIG. 6FIG. 6 is a diagrammatic perspective view illustrating a tracking element of the system in accordance with the invention, positioned on an articulating portion of a tibia, on the knee side.

FIG. 7FIG. 7 is a diagrammatic perspective view illustrating a guide block attached to a tibia and comprising a guide slot the position and/or orientation of which are adjustable relative to the guide block.

FIG. 8FIG. 8 is a diagrammatic perspective view of a sensor provided with a second three-dimensional visual marker.

FIG. 9FIG. 9 is a diagrammatic perspective view illustrating an instrument, such as a drill, provided with a three-dimensional visual marker.

### DETAILED DESCRIPTION OF THE INVENTION

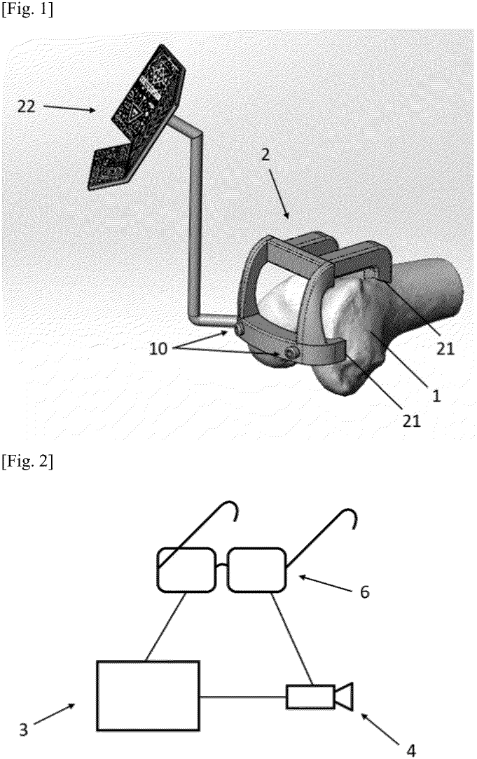

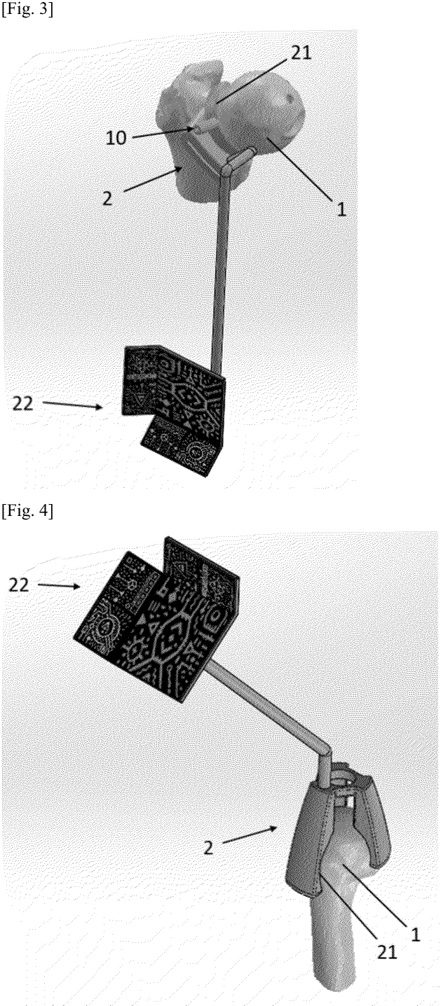

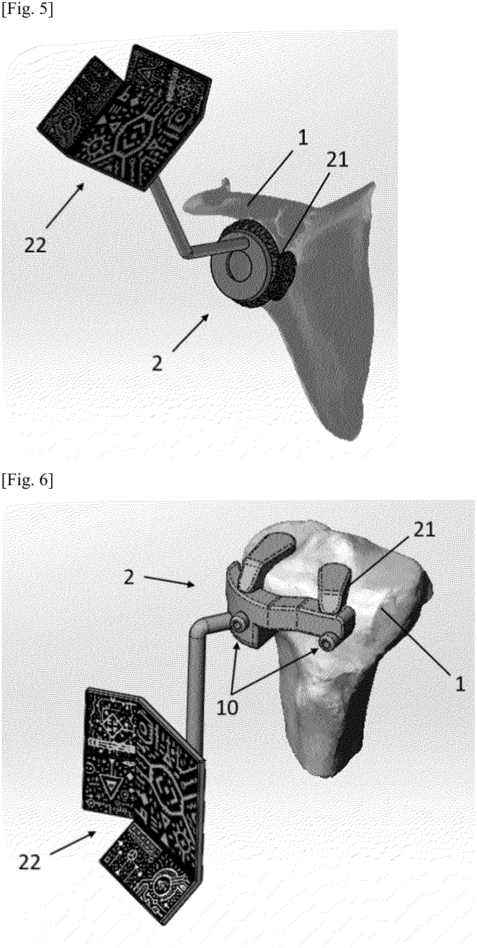

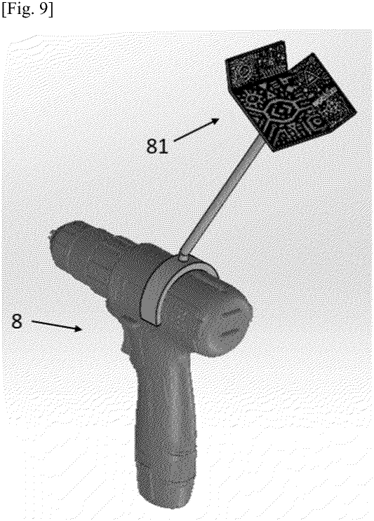

With reference to FIGS. 1 to 9, the invention concerns an augmented reality system for guiding a surgical operation on an articulating portion (1) of a bone.

The invention is not limited to a particular joint, and makes it possible to guide the positioning, i.e. the production of a bone cut or drilling, of any type of orthopaedic joint implant, such as a knee, shoulder or hip prosthesis, etc.

In known manner, the guidance system comprises a tracking element (2) intended to be secured to an articulating portion (1) of a bone. The tracking element (2) comprises at least one face (21) bearing on the articulating portion, made to measure from a virtual bone model.

This makes it possible to create a unique relationship between the virtual bone model and the tracking element (2) in a manner such that if the position and orientation in space of the tracking element (2) are known, the position and orientation in space of the virtual bone model are known extremely accurately.

In order to be able to know the position and orientation in space of the tracking element (2), in known manner, this comprises a three-dimensional visual marker (22) integrating information relating to the position and orientation of the tracking element (2) which is capable of being processed by a processing system (3), in particular a computer system.

In known manner again, the augmented reality system comprises a camera (4) intended to display and to track the three-dimensional visual marker (22) of the tracking element (2), and a processing system (3) configured to process data from the virtual bone model and, in real time, image data obtained from the camera (4) in order to determine the position and the orientation of the tracking element (2).

In a manner known to the person skilled in the art, the camera (4) is firmly attached, in particular in terms of a frame of reference, to a display unit (6) making it possible to display information transmitted by the processing system (3), superimposed on the field of view of a user.

The display unit (6) is, for example, in the form of a screen enabling the images obtained from the camera (4) to be visualized directly and the information transmitted by the processing system (3) to be superimposed thereon, or alternatively it is in the form of augmented reality spectacles, i.e. spectacles comprising at least one lens forming the display unit (6) and through which the user can observe a scene, and onto which the display unit (6) can superimpose the information.

In the latter embodiment, and in a known manner, the augmented reality spectacles may also carry the camera (4).

In this manner, when the processing system (3) detects that the camera (4) is visualizing the three-dimensional visual marker (22) of the tracking element (2), the processing system (3) displays on the display unit (6) a three-dimensional virtual representation of at least one geometric datum relating to the bone model positioned relatively with respect to the tracking element (2). In other words, the virtual representation of the geometric datum is attached to the tracking element (2), in accordance with a single position and orientation which in particular depends on those of said tracking element (2).

The geometric datum relating to the bone model represented virtually and displayed on the display unit (6) may be of any type, such as a geometrical surface or contours of the bone model, a mechanical axis, planned cutting planes or drilling axes, etc.

The augmented reality system is well known in the art and will not be described in further detail. The invention in particular consists in correcting the inaccuracy of this system, which is inherent to the presence of cartilage or fat on the articulating portion to which the tracking element (2) is to be secured.

To accomplish this, the system in accordance with the invention comprises a sensor (7) also provided with a three-dimensional visual marker (71) carrying information relating to the position and the orientation of the sensor (7) in space.

When the tracking element (2) is secured to the actual bone, the display unit (6) will virtually represent the geometric datum relating to the bone model superimposed on the actual scene. However, given the presence of cartilage, there will be a slight discrepancy between the real image of the bone and the virtual representation. In fact, and in practice, the tracking element (2) is not directly in contact with the bone, but in contact with an extra thickness of cartilage.

To correct this discrepancy, the surgeon will palpate at least three zones with the marked sensor (7), for example three zones of approximately 2 mm² which are also non-cartilaginous.

Since the processing system (3) accurately determines the position and orientation of the elements secured to the markers (22, 71), the position and spatial orientation of the sensor (7) with respect to the position and orientation of the tracking element (2) is known.

In theory, the three zones which the sensor (7) will palpate must normally be zones which correspond to equivalent zones of the virtual bone model. The processing system (3) is configured to rectify this discrepancy and very precisely reposition the virtual bone model, and in particular the virtual representation of the geometric datum, with respect to the three-dimensional visual marker (22) of the tracking element (2), and therefore with respect to the tracking element (2) itself.

From the foregoing, this makes it possible to know the position of the articulating portion of the bone very precisely, and to visualize it in three dimensions and virtually on the display unit (6) superimposed on the real bone. It is also possible to visualize the mechanical axis of the bone, or in fact the planned cutting planes or drilling axes.

In order to then facilitate and guide the surgical operation, the system in accordance with the invention also comprises an instrument (8), such as a cutting or drilling instrument, which also comprises a three-dimensional visual marker (81) carrying information relating to the position and the orientation of said instrument (8) in the space which the processing system (3) is capable of processing when it is visualized by the camera (4).

In other words, it is possible to know the position and the orientation of the instrument (8) precisely with respect to the visual marker (22) of the tracking element (2), and therefore with respect to the bone itself. The information integrated into the three-dimensional visual marker (81) of the instrument (8) enables the processing system (3) to display a drilling axis or a cutting plane linked to the instrument (8), for example, on the display unit (6) and superimposed on the field of vision of the surgeon, which is perfectly positioned with respect to the real bone, without inaccuracy relating to the extra thickness of cartilage.

The tracking element (2) may also comprise means for securing a guide block (9), in particular in a known and determined position and orientation relative to the tracking element (2).

In this configuration, the three-dimensional visual marker (22) of the tracking element (2) may be attached to the tracking element (2) in a removable or separable manner, allowing the surgeon to remove it so as not to interfere with the subsequent drilling cutting operation.

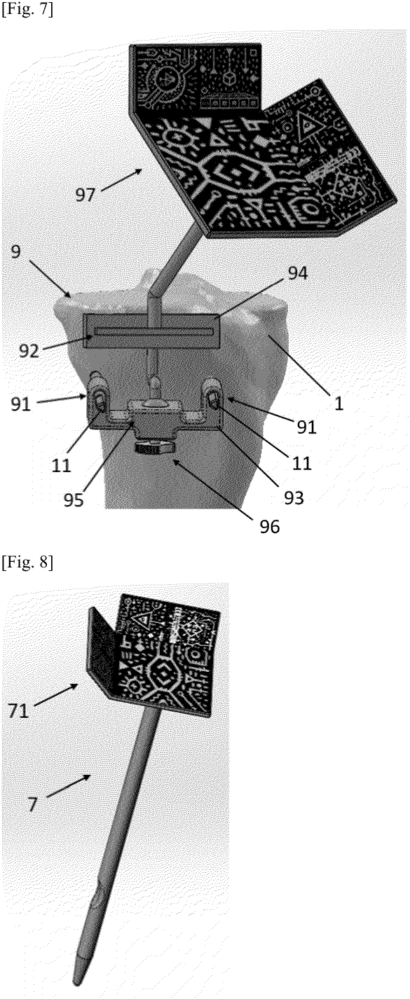

In accordance with another embodiment, the tracking element (2) comprises at least one, and preferably two orifices (10) adapted to receive two pins (11) intended to be secured in the bone. The tracking element (2) may then be removed by sliding it along the pins (11), and a guide block (9) also comprising two identical orifices (91) may be secured to the bone by positioning it by means of the two pins (11), see FIG. 7. The guide block (9) comprises a slot (92) and/or guide orifice the position and orientation of which are known with respect to the bone.

In order to be able to adapt to the surgeon’s requirements, the position and/or orientation of the slots (92) or guide orifice are adjustable with respect to the guide block (9).

As an example, with reference to FIG. 7, the guide block (9) comprises a principal portion (93) intended to be secured to the bone by the securing pins (11), and a guide slot (92) formed in a secondary portion (94) of the block, connected to the principal portion via a ball joint (95). Means for clamping (96), for example by screwing, are secured to the ball joint (95) to prevent it from pivoting. The secondary portion (94) of the guide block (9) comprises a three-dimensional visual marker (97) carrying information relating to the position and the orientation of the slot (92) in space which the processing system (3) is capable of processing when it is visualized by the camera (4).

In other words, it is possible to know the position and the orientation of the slot (92) precisely with respect to the visual marker (22) of the tracking element (2), and therefore with respect to the bone itself. The information integrated into the three-dimensional visual marker (97) of the secondary portion (94) of the guide block (9) enables the processing system (3) to display a cutting plane or drilling axis linked to the secondary portion (94) of the guide block (9) on the display unit (6), for example, which is superimposed on the field of vision of the surgeon and perfectly positioned with respect to the real bone, without inaccuracy relating to the extra thickness of cartilage.

1. An augmented reality system for guiding a surgical operation on an articulating portion of a bone, the system comprising:
a tracking element configured to be attached to the articulating portion of the bone, the tracking element comprising, on the one hand, at least one face for bearing on the articulating portion made to measure from a virtual model of the articulating portion of the bone and, on the other hand, a three-dimensional visual marker
a camera for visualizing and tracking the marker
a processing system configured to process data from the virtual bone model and, in real time, image data obtained from the camera in order to determine the position and orientation of the tracking element (2), in such a manner as to display, on a display unit secured to the camera and superimposed on the field of view of a user, a three-dimensional virtual representation of at least one geometric datum relating to the bone model the position and orientation of which are determined by the position and orientation of the tracking element
wherein theguidance system comprises a sensor which is also provided with a three-dimensional visual marker and to palpate at least three zones of a portion of the bone outside the cartilage zone, the marker of the sensor also being be tracked by the camera, and the processing system being further configured in order to determine the position and the orientation of the sensor with respect to the position and the orientation of the tracking element in a manner such as to correct, on the display unit and after palpation, the position and the orientation of the three-dimensional virtual representation of the geometric datum relating to the bone model with respect to the tracking element .

2. The guidance system as claimed in claim 1, comprising a cutting or drilling instrument provided with a three-dimensional visual marker, the markerbeing configure to be tracked by the camera and the processing systembeing further configured in order to determine the position and the orientation of the instrument with respect to the position and the orientation of the tracking element in a manner such as to display, on the display unit the position and the orientation of a cutting plane or drilling axis linked to the instrument with respect to the tracking element.

3. The guidance system as claimed in claim 1, the display unit is a screen displaying the images obtained from the camera.

4. The guidance system as claimed in claim 1, whereinthe display unit is at least one spectacle lens configured to be worn by the user.

5. The guidance system as claimed in claim 4, whereinthe at least one spectacle lens carries the camera.

6. The guidance system as claimed in claim 1, wherein the geometric datum relating to the bone model corresponds to the external surface of said virtual bone model, and the processing system is further configured, after correction, to process the data for the virtual bone model in order to increase its dimensions at the level of the bearing surface of the tracking element so that the display unit represents the bearing face of the tracking element in contact with the external surface of the virtual bone model.

7. The guidance system as claimed in claim 1, whereinthe visual marker is attached to the tracking element in a removable or separable manner.

8. The guidance system as claimed in claim 1, wherein the tracking element comprises means for securing a guide block.

9. The guidance system as claimed in claim 1, wherein the tracking element comprises at least one orifice for guiding a pin for securing to the bone, and the system comprises a guide block having at least one orifice for the passage of the pin the guide block being to be secured to the bone in a position and at an orientation which is determined and guided by the pin.

10. The guidance system as claimed in claimwherein guide block comprises at least one slot and/or at least one guide orifice the position and/or orientation of which can be adjusted with respect to the guide block.

11. The guidance system as claimed in claim 9, wherein the guide block comprises at least one slot and/or at least one guide orifice the position and/or orientation of which can be adjusted with respect to the guide block.

12. The guidance system as claimed in claim 1, comprising a pin configured to secure a guide block to the bone.

13. The guidance system as claimed in claim 12, wherein the guide block comprises at least one slot and/or at least one guide orifice the position and/or orientation of which can be adjusted with respect to the guide block.
